(19) United States
(12) Patent Application Publication
(10) Pub. No.: US 2012/0263780 A1
(43) Pub. Date: Oct. 18, 2012

(54) Carbohydrate Entrapped Active Agent Delivery Composition and Articles Using the Same

(76) Inventors: Scott W. Wenzel, Neenah, WI (US); David William Koenig, Menasha, WI (US); Jeffery Richard Seidling, Appleton, WI (US); Michael Meihan Chen, Appleton, WI (US)

(21) Appl. No.: 13/534,028

(22) Filed: Jun. 27, 2012

Related U.S. Application Data
(62) Division of application No. 12/578,348, filed on Oct. 13, 2009.

(51) Int. Cl. A01N 31/08; A61K 31/05; A61P 29/00; A01P 1/00; A61P 31/00; A61P 35/00; A61K 9/70; A01N 25/08

(52) U.S. Cl. 424/443; 514/731

(57) ABSTRACT

Delivery compositions and substrates for imparting a volatile active agent to a surface are disclosed herein. To achieve the delivery of the volatile component, a delivery composition of a carbohydrate matrix and a volatile component is incorporated into the carbohydrate matrix to stabilize the volatile for extended periods of time. The delivery composition further comprises a self-generating expelling agent to expel the volatile component onto the contaminated surface or skin upon addition of a triggering agent.

### PRIORITY

This application is a divisional of application Ser. No. 12/578,348 entitled Carbohydrate Entrapped Active Agent Delivery Composition and Articles Using the Same and filed in the U.S. Patent and Trademark Office on Oct. 13, 2009. The entirety of the prior application is hereby incorporated by reference in this application.

### BACKGROUND

Cleansing household and personal care products have been used for numerous years. In recent years, however, consumers have begun demanding more out of household and personal care products. For example, a new trend has emerged in the consumer market where consumers are expressing a growing demand for “natural” products. These consumers are willing to pay a premium for these natural compounds but finding natural compounds that are safe, efficacious and stable presents a problem. Additionally, consumers want products that perform more tasks, such as cleaning or disinfecting, better or more efficiently.

Currently, one such natural active agent that has been identified to provide such is thymol, which is an active constituent of thyme. Thyme contains a large concentration of volatile oil. The primary component of that oil is thymol. Thymol, a monoterpene phenol derivative of cymene, is extracted from the culinary plant thyme as a white substance of pleasant aromatic odor and strong antiseptic properties. The potent antimicrobial activity and origins of this compound make it ideal for disinfectant applications. Thymol is EPA-registered disinfectant and is used with various consumer products. It is also commonly used to treat fungal infections such as athlete's foot. Thymol is an effective antimicrobial agent with proven efficacy against yeast, mold and mycobacteria.

With all of its positive attributes however, a problem exists with thymol and other active agents. Many active agents such as thymol are extremely volatile compounds that are not easily stabilized and which tend to flash or volatilize off the substrate and lose efficacy over time. Use of volatile active agents on a dry wipe typically show no efficacy as the active agent volatilized off the substrate and was unavailable for efficacious action.

Therefore, a need exists for a delivery composition that will stabilize and allow the incorporation of volatile active agents. A need also exists to provide a substrate with a delivery composition incorporated within to provide the beneficial effect of the volatile active agents.

### SUMMARY

Generally, compositions and substrates for imparting a volatile active agent to a surface are disclosed herein. The delivery composition for delivering the active agent includes a carbohydrate matrix, a volatile active agent entrapped within the carbohydrate matrix, and an expelling agent entrapped within the carbohydrate matrix.

The volatile active agent may be selected from disinfectants, antimicrobials, anti-proliferative agents, and anti-inflammatory agents. Desirably, the volatile active agent is thymol. The active agent may be present in an amount from about 0.05% by weight of the delivery composition to about 5% by weight of the delivery composition.

The carbohydrate matrix may be formed from a simple or complex carbohydrate selected from glucose, fructose, sucrose, lactose, maltose, isomaltose, corn syrup, and mixtures thereof.

The expelling agent may be a gas selected from carbon dioxide, nitric oxide or nitrogen. Alternatively, the expelling agent is a triggerable expelling agent selected from citric acid and bicarbonate that is triggered by water to produce a gas. The expelling agent provides a cue to consumers by cracking, snapping, popping, tingling, or tickling which provides reinforcement that the product is working. The expelling agent may be present in an amount of from about 0.1 to 20 cm³ of gas per gram of the carbohydrate matrix. Desirably, the expelling agent is present in an amount of from about 1 to 10 cm³.

Desirably, the delivery composition may be formulated within an anhydrous carrier.

The delivery composition may further contain a trigger control agent selected from gums, cellulosics, starches, clays, acrylate based agents, colloidal, fatty acids and their salts, fatty alcohols, fatty esters, butters, natural waxes, synthetic waxes, silicone waxes, silicone crosspolymers, or beeswax, and mixtures thereof.

The delivery composition may also include a colorant entrapped within the carbohydrate matrix, the colorant selected from FD&C/D&C approved pigments, dyes, lakes and natural colorants and extracts to provide an additional visual cue to the user.

The delivery composition may further contain a skin benefit agent selected from the group consisting of a surfactant, a quaternary ammonium material, a particulate, a rheology modifier, a humectant, a moisturizer, a film former, a slip modifier, a surface modifier, a skin protectant, a sunscreen, and combinations thereof.

The delivery composition may also be present on a substantially dry substrate to deliver the active agent. The dry substrate may be selected from facial tissue, bath tissue, paper towel, or dinner napkin.

### BRIEF DESCRIPTION OF THE DRAWINGS

The above aspects and other features, aspects, and advantages of the present invention will become better understood with regard to the following description, appended claims, and accompanying drawings in which:

FIG. 1 is a perspective view of one embodiment of a dry substrate product made with the delivery composition described herein.

FIG. 2 is a cross-sectional view of the dry substrate product illustrated in FIG. 1.

FIG. 3 is a cross-sectional view of another embodiment of a dry substrate product made with the delivery composition described herein.

FIG. 4 is a perspective view of one embodiment of a spirally wound paper towel product with the delivery composition described herein.

Repeated use of reference characters in the specification and drawings is intended to represent the same or analogous features or elements of the invention in different embodiments.

### DETAILED DESCRIPTION

It is to be understood by one of ordinary skill in the art that the present discussion is a description of exemplary embodiments only, and is not intended as limiting the broader aspects of the present invention, which broader aspects are embodied in the exemplary construction.

Generally, delivery compositions and substrates for imparting a volatile active agent to a surface are disclosed herein. To achieve the delivery of the volatile component, a delivery composition may contain a carbohydrate matrix and a volatile component incorporated into the carbohydrate matrix to stabilize the volatile component for extended periods of time. The delivery composition further comprises a self-generating expelling agent to expel the volatile component to the contaminated surface upon addition of a triggering agent.

An operative delivery composition contains a carbohydrate matrix, an effective amount of at least one active agent and an effective amount of at least one expelling agent. The carbohydrate matrix would dissolve when added to a triggering agent, such as water, alcohol, glycols such as propylene, butylene, glycerin, and glycol derivatives such as methyl gluceth-10, methyl gluceth-20 to trigger and release the expelling agent which in turn would spray an effective amount of active agent onto the desired region of a surface or skin.

Typically, the carbohydrate matrix is a made from a simple or complex sugar or other carbohydrate. Various examples of carbohydrates for use with the delivery composition include, but are not limited to, glucose, fructose, sucrose, lactose, maltose, isomaltose, corn syrup, and mixtures thereof. The carbohydrate matrix has the ability to entrap, encapsulate and/or stabilize an active agent for extended periods of time such that it will be active when released from the matrix. To trigger the release of the contents of the carbohydrate matrix, a triggering agent must be present. For example, the carbohydrate matrix dissolves in the triggering agent, such as water, to release its contents.

Typically, the delivery compositions comprise a simple or complex sugar or other carbohydrate making the carbohydrate matrix in an amount of from about 40% by weight of the delivery composition to about 99.9% by weight of the delivery composition, more typically from about 75% by weight of the delivery composition to about 99.9% by weight of the delivery composition, and more typically from about 90% by weight of the delivery composition to about 99% by weight of the delivery composition.

As described above, the carbohydrate matrix encapsulates a volatile active agent. In exemplary embodiments, the active agents disposed within the carbohydrate matrix are disinfectants, antimicrobials, anti-proliferative agents, anti-inflammatory agents that could be directed to combat bacteria, viruses, and/or fungi, or treat another medical condition. Suitable volatile active agents include, but are not limited to, essential oils, alcohols, and retinoids.

Desirably, the active agent may be an essential oil derived from 100% natural fats and oils that are derived from natural plant sources. Suitable natural fats or oils can include citrus oil, olive oil, avocado oil, apricot oil, babassu oil, borage oil, camellia oil, canola oil, castor oil, coconut oil, corn oil, cottonseed oil, evening primrose oil, green tea oil, hydrogenated cottonseed oil, hydrogenated palm kernel oil, jojoba oil, maleated soybean oil, meadowfoam seed oil, palm kernel oil, peanut oil, rapeseed oil, grapeseed oil, safflower oil, sweet almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandalwood oil, tea tree oil, sunflower oil, soybean oil, thyme oil, peppermint oil, spearmint oil, basil oil, anise oil, menthol, camphor, turpentine oil, ylang ylang oil, rosemary oil, lavender oil, sandalwood oil, cinnamon oil, marojoram oil, cajuput oil, lemongrass oil, orange oil, grapefruit oil, lemon oil, fennel oil, ginger oil, marjoram oil, pine oil, clove oil, oregano oil, rosewood oil, sage oil, parsley oil, myrrh oil, mugwort oil, elderberry oil, cedarwood oil, and combinations thereof.

More desirably, the active agent may be the volatile disinfectant, thymol. Thymol is an effective antimicrobial agent with proven efficacy against yeast, mold and mycobacteria.

Other active agents that may also be useful with the delivery agent include, but are not to be limited to, a-pinene, b-pinene, sabinene, myrcene, a-phellandrene, a-terpinene, limonene, 1,8-cineole, y-terpinene, p-cymene, terpinolene, linalool, terpinen-4-ol, a-terpineol, carvone, myrcene, caryophyllene, menthol, citronellal, geranyl acetate, nerol, geraniol, neral, citral, and combinations thereof.

An effective amount of an active agent would be at an amount necessary within the composition to produce the desired end benefit upon delivery to the surface. Typically, the delivery compositions comprise the active agent in an amount of from about 0.01% by weight of the delivery composition to about 5.0% by weight of the delivery composition, more typically from about 0.01% by weight of the delivery composition to about 4.0% by weight of the delivery composition, and more typically from about 0.01% by weight of the delivery composition to about 3.0% by weight of the delivery composition.

As described above, the composition further comprises an expelling agent to expel the volatile active agent to the contaminated surface upon addition of a triggering agent. An expelling agent is an ingredient that has the ability to spray, expel, propel and/or dispense the active agent from the matrix when the matrix substance dissolves in water. By spraying the active agent from the matrix, the expelling agent increases the area of the skin or surface that is treated with the active agent.

The expelling agent may be self-generating and can take the form of a gas such as carbon dioxide, nitric oxide, nitrogen, air, or other entrapped gases that are entrapped within the carbohydrate matrix. The gases are preferably substantially unreactive with the carbohydrate matrix. Upon triggering of the encapsulating carbohydrate matrix, the expelling agent is released causing a sparkling, popping action. The cracking, snapping, popping, tingling, or tickling provides reinforcement that the product is working by providing a cue to consumers.

Alternatively, the expelling agent may be a triggerable ingredient that will produce a gas when placed in contact with the triggering agent. For example, non-limiting examples of the expelling agent may be citric acid or sodium bicarbonate. When the expelling agent, citric acid or sodium bicarbonate, comes into contact with water as the triggering agent, the expelling agent will react with the water and from a gas.

Typically, the delivery compositions comprise the expelling agent in an amount of from about 0.1 to 20 cm³ of gas per gram of the carbohydrate matrix. Desirably, the expelling agent is present in an amount of from about 1 to 10 cm³.

To produce an exemplary delivery composition, a sugar is heated to make a molten slurry of sugar upon which an active agent, thymol, is added in an enclosed system. The sugar/active agent solution is then cooled and as it is cooled, an expelling agent, carbon dioxide, is mixed into the slurry to become an entrapped gas within the matrix. The final product once cooled appears as a crystalline powder which contains the entrapped gas and the active agent. This powder could be shaken on the surface of the skin or countertop and wetted.

Upon triggering of the crystalline delivery composition, the sugar melts and releases the expelling agent, carbon dioxide. The release of the expelling agent sprays the active agent onto the surface of the skin or countertop. In addition to spraying the active agent, the expelling agent also serves a sensory benefit in that it provides a cue to consumers by cracking, snapping, popping, tingling, or tickling which provides reinforcement that the product is working.

The delivery composition may also include a colorant entrapped within the carbohydrate matrix. The colorant could include but not be limited to FD&C/D&C approved pigments, dyes, or lakes. The colorant could also include, but not be limited to natural colorants and extracts such as walnut extract, chicory extract, beet extract, carmine, and henna. Upon addition of water, the sugar matrix dissolves releasing the colorant and expelling agent to provide both a visual (colorant) and a sensory (crackling, popping, tickling) cue to the consumer.

The delivery composition may also contain a trigger control agent to control the speed at which the delivery vehicle is dissolved or to control the rate at which the expelling agent and active agent are delivered from the delivery composition. Trigger control agents could include, but are not limited to, a trigger control agent selected from gums, cellulosics, starches, clays, acrylate based agents, colloidal (fumed silica, gel silica), fatty acids and their salts, fatty alcohols, fatty esters, butters, natural waxes (vegetable derived), synthetic waxes (petroleum derived), silicone waxes, silicone crosspolymers, or beeswax such that the delivery composition is substantially thickened and the expelling agent, active agent and colorant are adequately suspended throughout the carbohydrate matrix. The trigger control agent acts to slow the release of the composition when water is used as the triggering agent.

Various other agents could be incorporated within this composition to facilitate the expelling process, converting process, stability of the composition, etc. and these various other agents are known in the art.

For example, an accelerant may be used to facilitate quicker utilization of the active agent by the user like allowing the active agent to penetrate the skin faster. Suitable accelerants include, but are not limited to, glycols such as propylene, butylene, hexylene, pentylene, caprylyl, and the like; glycol derivatives such as ethoxydiglycol, ethylhexyl glycerin, glyceryl caprylate, glyceryl caprate; dimethyl isosorbide, and combinations thereof.

An effective amount of an accelerant would be at an amount necessary within the composition to produce the desired end benefit by facilitating quicker utilization of the active agent by the user, such as allowing the active agent to penetrate the skin faster. Typically, the delivery compositions comprise the accelerant in an amount of from about 0.01% by weight of the delivery composition to about 10.0% by weight of the delivery composition, more typically from about 0.1% by weight of the delivery composition to about 10.0% by weight of the delivery composition.

Other suitable agent(s) would allow for easier metering of a very small quantity of active agents unto the product when the composition is placed on the product during converting.

The delivery composition may be formulated with one or more conventional pharmaceutically-acceptable and compatible carrier materials to form a personal care delivery composition. The personal care delivery composition may take a variety of forms including, without limitation, aqueous solutions, gels, balms, lotions, suspensions, creams, milks, salves, ointments, sprays, foams, solid sticks, aerosols, and the like. The carrier is preferably anhydrous such that the carrier has typically less than 15% water present, more typically less than 10% water present, and even more typically less that 5% water present. Use of an anhydrous carrier avoids activating the water-triggerable matrix and releasing the active agents or expelling agents entrapped therein. The anhydrous carrier could include, but not be limited to, one or blends of the following ingredient types: fatty acids, fatty alcohols, surfactants, emollients, moisturizers, humectants, natural oils (vegetable derived), synthetic oils (petroleum derived), silicone oils, cosmetic emollient oils (including esters, ethers, hydrocarbons, etc.) as described below.

Examples of such suitable agents include emollients, sterols or sterol derivatives, natural and synthetic fats or oils, viscosity enhancers, rheology modifiers, polyols, surfactants, alcohols, esters, silicones, clays, starch, cellulose, particulates, moisturizers, film formers, slip modifiers, surface modifiers, skin protectants, humectants, sunscreens, and the like.

Thus, the delivery compositions may further optionally include one or more emollient, which typically acts to soften, soothe, and otherwise lubricate and/or moisturize the skin. Suitable emollients that can be incorporated into the compositions include oils such as petrolatum based oils, natural oils, petrolatum, mineral oils, alkyl dimethicones, alkyl methicones, alkyldimethicone copolyols, phenyl silicones, alkyl trimethylsilanes, dimethicone, dimethicone crosspolymers, cyclomethicone, lanolin and its derivatives, glycerol esters and derivatives, propylene glycol esters and derivatives, alkoxylated carboxylic acids, alkoxylated alcohols, and combinations thereof.

Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof, can also suitably be used as emollients.

The delivery composition may include one or more emollient in an amount of from about 0.01% by weight of the delivery composition to about 70% by weight of the delivery composition, more desirably from about 0.05% by weight of the delivery composition to about 50% by weight of the delivery composition, and even more desirably from about 0.10% by weight of the delivery composition to about 40% by weight of the delivery composition. In instances wherein the composition is used in combination with a wet wipe, the composition may include an emollient in an amount of from about 0.01% by weight of the delivery composition to about 20% by weight of the delivery composition, more desirably from about 0.05% by weight of the delivery composition to about 10% by weight of the delivery composition, and more typically from about 0.1% by weight of the delivery composition to about 5.0% by weight of the delivery composition.

Optionally, one or more viscosity enhancers may be added to the personal care composition to increase the viscosity, to help stabilize the composition, such as when the composition is incorporated into a personal care product, thereby reducing migration of the composition and improve transfer to the skin. Suitable viscosity enhancers include polyolefin resins, lipophilic/oil thickeners, polyethylene, silica, silica silylate, silica methyl silylate, colloidal silicone dioxide, cetyl hydroxy ethyl cellulose, other organically modified celluloses, PVP/decane copolymer, PVM/MA decadiene crosspolymer, PVP/eicosene copolymer, PVP/hexadecane copolymer, clays, carbomers, acrylate based thickeners, surfactant thickeners, and combinations thereof.

The delivery composition may desirably include one or more viscosity enhancers in an amount of from about 0.01% by weight of the delivery composition to about 25% by weight of the delivery composition, more desirably from about 0.05% by weight of the delivery composition to about 10% by weight of the delivery composition, and even more desirably from about 0.1% by weight of the delivery composition to about 5% (by weight of the delivery composition.

The delivery composition may optionally further contain rheology modifiers. Rheology modifiers may help increase the melt point viscosity of the composition so that the composition readily remains on the surface of a personal care product.

Suitable rheology modifiers include combinations of alpha-olefins and styrene alone or in combination with mineral oil or petrolatum, combinations of di-functional alpha-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n-polymers, (styrene-isoprene) n-polymers, styrene-butadiene copolymers, and styrene-ethylene/propylene copolymers and combinations thereof. Specifically, rheology enhancers such as mineral oil and ethylene/propylene/styrene copolymers, and mineral oil and butylene/ethylene/styrene copolymers are particularly desirable.

The delivery composition can suitably include one or more rheology modifier in an amount of from about 0.1% by weight of the delivery composition to about 5% by weight of the delivery composition.

The delivery composition may optionally further contain humectants. Examples of suitable humectants include glycerin, glycerin derivatives, sodium hyaluronate, betaine, amino acids, glycosaminoglycans, honey, sorbitol, glycols, polyols, sugars, hydrogenated starch hydrolysates, salts of PCA, lactic acid, lactates, and urea. A particularly preferred humectant is glycerin. The delivery composition may suitably include one or more humectants in an amount of from about 0.05 by weight of the delivery composition to about 25% by weight of the delivery composition.

The delivery composition of the disclosure may optionally further contain film formers. Examples of suitable film formers include lanolin derivatives (e.g., acetylated lanolins), superfatted oils, cyclomethicone, cyclopentasiloxane, dimethicone, synthetic and biological polymers, proteins, quaternary ammonium materials, starches, gums, cellulosics, polysaccharides, albumen, acrylates derivatives, IPDI derivatives, and the like. The composition of the present disclosure may suitably include one or more film former in an amount of from about 0.01% by weight of the delivery composition to about 20% by weight of the delivery composition.

The delivery composition may optionally further contain slip modifiers. Examples of suitable slip modifiers include bismuth oxychloride, iron oxide, mica, surface treated mica, ZnO, ZrO₂, silica, silica silyate, colloidal silica, attapulgite, sepiolite, starches (i.e. corn, tapioca, rice), cellulosics, nylon-12, nylon-6, polyethylene, talc, styrene, polystyrene, polypropylene, ethylene/acrylic acid copolymer, acrylates, acrylate copolymers (methylmethacrylate crosspolymer), sericite, titanium dioxide, aluminum oxide, silicone resin, barium sulfate, calcium carbonate, cellulose acetate, polymethyl methacrylate, polymethylsilsequioxane, talc, tetrafluoroethylene, silk powder, boron nitride, lauroyl lysine, synthetic oils, natural oils, esters, silicones, glycols, and the like. The composition of the present disclosure may suitably include one or more slip modifier in an amount of from about 0.01% by weight of the delivery composition to about 20% by weight of the delivery composition.

The delivery composition may also further contain surface modifiers. Examples of suitable surface modifiers include silicones, quaternium materials, powders, salts, peptides, polymers, clays, and glyceryl esters. The composition of the present disclosure may suitably include one or more surface modifier in an amount of from about 0.01% by weight of the delivery composition to about 20% by weight of the delivery composition.

The delivery composition may also further contain skin protectants. Examples of suitable skin protectants include ingredients referenced in SP monograph (21 CFR part 347). Suitable skin protectants and amounts include those set forth in SP monograph, Subpart B—Active Ingredients Sec 347.10: (a) Allantoin, 0.5 to 2%, (b) Aluminum hydroxide gel, 0.15 to 5%, (c) Calamine, 1 to 25%, (d) Cocoa butter, 50 to 100%, (e) Cod liver oil, 5 to 13.56%, in accordance with 347.20(a)(1) or (a)(2), provided the product is labeled so that the quantity used in a 24-hour period does not exceed 10,000 U.S.P. Units vitamin A and 400 U.S.P. Units cholecalciferol, (f) Colloidal oatmeal, 0.007% minimum; 0.003% minimum in combination with mineral oil in accordance with §347.20(a)(4), (g) Dimethicone, 1 to 30%, (h) Glycerin, 20 to 45%, (i) Hard fat, 50 to 100%, (j) Kaolin, 4 to 20%, (k) Lanolin, 12.5 to 50%, (l) Mineral oil, 50 to 100%; 30 to 35% in combination with colloidal oatmeal in accordance with §347.20(a)(4), (m) Petrolatum, 30 to 100%, (o) Sodium bicarbonate, (q) Topical starch, 10 to 98%, (r) White petrolatum, 30 to 100%, (s) Zinc acetate, 0.1 to 2%, (t) Zinc carbonate, 0.2 to 2%, (u) Zinc oxide, 1 to 25%.

The delivery composition may also further contain sunscreens. Examples of suitable sunscreens include aminobenzoic acid, avobenzone, cinoxate, dioxybenzone, homosalate, menthyl anthranilate, octocrylene, octinoxate, octisalate, oxybenzone, padimate O, phenylbenzimidazole sulfonic acid, sulisobenzone, titanium dioxide, trolamine salicylate, zinc oxide, and combinations thereof. Other suitable sunscreens and amounts include those approved by the FDA, as described in the Final Over-the-Counter Drug Products Monograph on Sunscreens (Federal Register, 1999:64:27666-27693), herein incorporated by reference, as well as European Union approved sunscreens and amounts.

The delivery composition may also further contain quaternary ammonium materials. Examples of suitable quaternary ammonium materials include polyquaternium-7, polyquaternium-10, benzalkonium chloride, behentrimonium methosulfate, cetrimonium chloride, cocamidopropyl pg-dimonium chloride, guar hydroxypropyltrimonium chloride, isostearamidopropyl morpholine lactate, polyquaternium-33, polyquaternium-60, polyquaternium-79, quaternium-18 hectorite, quaternium-79 hydrolyzed silk, quaternium-79 hydrolyzed soy protein, rapeseed amidopropyl ethyldimonium ethosulfate, silicone quaternium-7, stearalkonium chloride, palmitamidopropyltrimonium chloride, butylglucosides, hydroxypropyltrimonium chloride, laurdimoniumhydroxypropyl decylglucosides chloride, and the like. The composition of the present disclosure may suitably include one or more quaternary material in an amount of from about 0.01% by weight of the delivery composition to about 20% by weight of the delivery composition.

The delivery composition may optionally further contain surfactants. Examples of suitable additional surfactants include, for example, anionic surfactants, cationic surfactants, amphoteric surfactants, zwitterionic surfactants, non-ionic surfactants, and combinations thereof. Specific examples of suitable surfactants are known in the art and include those suitable for incorporation into personal care compositions and wipes. The composition of the present disclosure may suitably include one or more surfactants in an amount of from about 0.01% by weight of the delivery composition to about 20% by weight of the delivery composition.

The delivery composition may also further contain additional emulsifiers. As mentioned above, the natural fatty acids, esters and alcohols and their derivatives, and combinations thereof, may act as emulsifiers in the composition. Optionally, the composition may contain an additional emulsifier other than the natural fatty acids, esters and alcohols and their derivatives, and combinations thereof. Examples of suitable emulsifiers include nonionics such as polysorbate 20, polysorbate 80, anionics such as DEA phosphate, cationics such as behentrimonium methosulfate, and the like. The composition of the present disclosure may suitably include one or more additional emulsifier in an amount of from about 0.01% by weight of the delivery composition to about 20% by weight of the delivery composition.

The delivery composition may additionally include adjunct components conventionally found in pharmaceutical compositions in their art-established fashion and at their art-established levels. For example, the compositions may contain additional compatible pharmaceutically active materials for combination therapy, such as antimicrobials, antioxidants, anti-parasitic agents, antipruritics, antifungals, antiseptic actives, biological actives, astringents, keratolytic actives, local anesthetics, anti-stinging agents, anti-reddening agents, skin soothing agents, and combinations thereof. Other suitable additives that may be included in the compositions of the present disclosure include colorants, deodorants, fragrances, perfumes, emulsifiers, anti-foaming agents, lubricants, natural moisturizing agents, skin conditioning agents, skin protectants and other skin benefit agents (e.g., extracts such as aloe vera and anti-aging agents such as peptides), solvents, solubilizing agents, suspending agents, wetting agents, humectants, preservatives, pH adjusters, buffering agents, dyes and/or pigments, and combinations thereof.

The delivery composition may also be used in combination with a product, such as a personal care product. More particularly, the carbohydrate entrapped volatile component may be incorporated into a composition that may be incorporated into or onto a substrate, such as a wipe substrate, an absorbent substrate, a fabric or cloth substrate, or a tissue substrate, among others. For example, the compositions may be incorporated into personal care products, such as wipes, absorbent articles, bath tissues, cloths, and the like. More particularly, the carbohydrate entrapped volatile component-containing composition may be incorporated into wipes such as wet wipes, dry wipes, hand wipes, face wipes, cosmetic wipes, and the like, or absorbent articles, such as paper towels, diapers, training pants, adult incontinence products, feminine hygiene products, and the like. Desirably, the carbohydrate entrapped volatile component may be incorporated onto a dry substrate. The dry substrate may be prepared by applying a composition containing a carbohydrate entrapped volatile component of the present disclosure onto a wipe substrate by any suitable means (e.g., by spraying, impregnating, etc.). The composition may comprise 100% carbohydrate entrapped volatile component, or alternately, the composition may be present in the composition in combination with a carrier and/or other skin benefit agent, as described herein. In embodiments where the composition used to prepare the dry wipe comprises water or moisture, the resulting treated substrate is then dried so that the wipe comprises less than about 10% by weight of the substrate moisture content, and a dry wipe is produced. The treated substrate can be dried by any means known to those skilled in the art including, for example by use of convection ovens, radiant heat sources, microwave ovens, forced air ovens, and heated rollers or cans, or combinations thereof.

As used interchangeably herein, the terms “dry substrate” and “substantially dry substrate” mean a wipe that includes less than about 10% by weight of the substrate moisture content. Specifically, a “dry substrate” can be a facial tissue, bath tissue, paper towel, dinner napkin, or the like. The tissue products of this invention can be one-ply, two-ply, three-ply or more. In all cases, the composition is applied to one or both outer surfaces of the product after the product has been dried. The composition can be applied after the plies are brought together or prior to bringing the plies together. The individual plies can be layered or blended (homogeneous) creped or uncreped, throughdried or wet-pressed. Alternately, materials suitable for the substrate of the wipes are well know to those skilled in the art, and are typically made from a fibrous sheet material which may be either woven or nonwoven. For example, suitable materials for use in the wipes may include nonwoven fibrous sheet materials which include meltblown, coform, air-laid, bonded-carded web materials, hydroentangled materials, and combinations thereof. Such materials can be comprised of synthetic or natural fibers, or a combination thereof. Typically, the wipes of the present disclosure define a basis weight of from about 25 grams per square meter to about 120 grams per square meter and desirably from about 40 grams per square meter to about 90 grams per square meter.

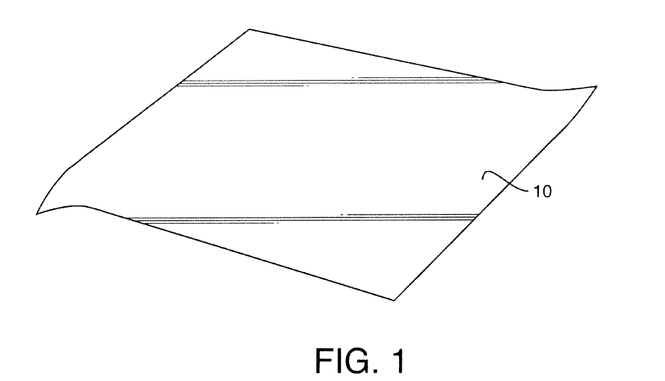

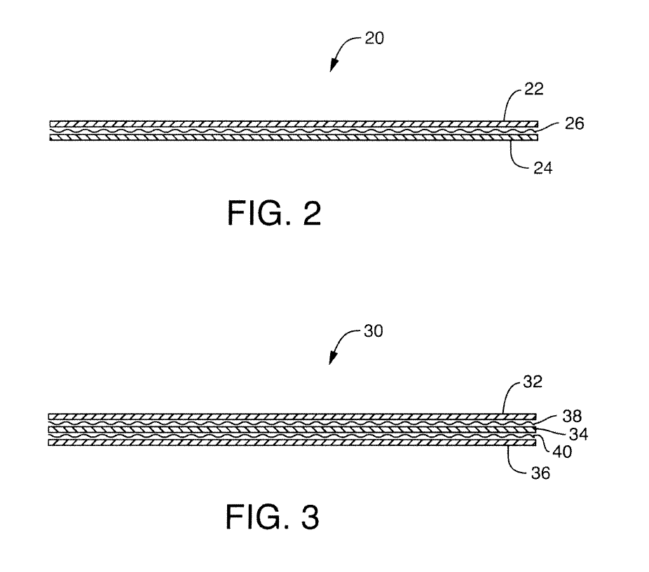

Specifically, the delivery composition can be present on an exterior surface of a tissue product 10 as shown on FIG. 1. Alternatively, the delivery composition can be incorporated into the tissue product in a manner so that substantially none of the delivery composition is present on the exterior surfaces. For instance, referring to FIG. 2, a tissue product 20 is shown that is comprised of a first tissue web 22 laminated to a second tissue web 24. As shown, positioned in between the first tissue web 22 and the second tissue web 24 is a delivery composition 26 made in accordance with the present disclosure. By locating the delivery composition 26 in between the tissue webs, the delivery composition is substantially prevented from being transferred to a user's skin. When the tissue product 20, however, is held against the skin, moisture from the skin will be absorbed by the delivery composition 26 through the tissue webs thus melting the carbohydrate matrix and releasing the active agent. The presence of the moisture will trigger a delivery to occur in the delivery composition 26 providing the activated response of the active agent to the skin of the user.

In one specific embodiment the product is a facial tissue comprising three or more plies, two outer plies and one or more interior plies. The delivery composition is applied to at least one of the one or more interior plies. In another embodiment, the tissue product is a facial tissue comprising two plies, comprising two outer facing surfaces and two oppositely facing inner surfaces. The delivery composition is applied to one or both of the oppositely facing inner surfaces. In another embodiment, the product is a multi-ply tissue product where the delivery composition is applied selectively to the inner portion of the multi-ply product so as to minimize blocking.

In this manner, other beneficial compositions may be applied to the exterior surface of the tissue product and used in conjunction with the delivery composition 26. For example, in one embodiment, a lotion that is intended to moisturize the skin can be present on at least one exterior surface of the tissue product and may work in conjunction with the delivery composition. In this manner, the tissue product 20 can not only provide an active agent to the user, but can also transfer a moisturizer to the skin.

In addition to lotions, any other suitable composition may also be applied to the exterior surface. For instance, in one embodiment, various softening agents may be present on the exterior surfaces of the tissue product. One example of a softening agent may comprise a polysiloxane.

In addition to a 2-ply product as shown in FIG. 2, other tissue products that may be made in accordance with the present disclosure can include more than two plies. For example, a 3-ply tissue product 30 is illustrated in FIG. 3. As shown, the tissue product 30 includes a middle tissue web 34 laminated to outer tissue webs 32 and 36. In accordance with the present disclosure, a delivery composition is located in between the first tissue web 32 and the middle tissue web 34. A delivery composition 40 is also positioned in between the middle tissue web 34 and the second outer tissue web 36.

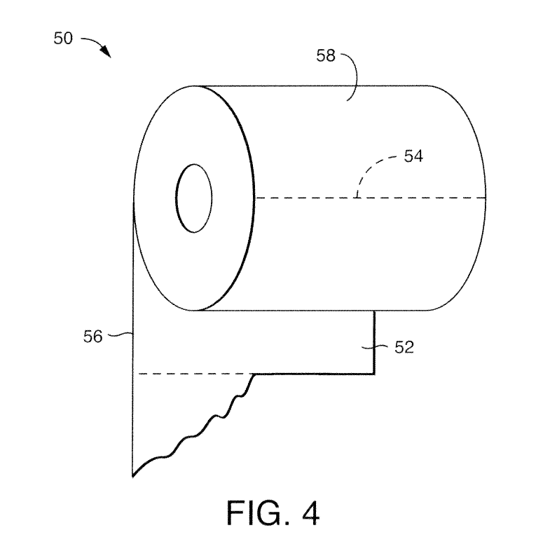

In an alternative embodiment, the delivery composition of the present disclosure can also be present on one or more exterior surfaces of a dry substrate product. For instance, referring to FIG. 4, in one embodiment the delivery composition can be applied to an exterior surface of a paper towel product 50. As shown, the paper towel product 50 comprises a spirally wound product containing individual tissue sheets 52 separated by perforation lines 54. The tissue sheets can include a first exterior surface 56 and a second exterior surface 58. Each tissue sheet may comprise a single ply product or a multi-ply product. In accordance with the present disclosure, the delivery composition may be present on the first exterior surface 56, on the second exterior surface 58, or on both exterior surfaces.

Applying the delivery composition to a paper towel product as shown in FIG. 4 may provide various unexpected benefits and advantages. Thus, the disinfectant is stored within the paper towel allowing for the consumer to disinfect the surface from harmful microorganisms. Furthermore, a more naturally derived chemistry such as thymol is perceived as safer and earth-friendly by consumers.

The dry substrate may comprise the composition in an add-on amount of composition of from about 40% by weight of the treated substrate to about 250% by weight of the treated substrate, more typically from about 75% by weight of the treated substrate to about 150% by weight of the treated substrate and more typically about 100% by weight of the treated substrate.

Upon wetting the substantially dry substrate or upon contact with water, such as a wet countertop or skin, the carbohydrate entrapped volatile component-containing composition dissolves to release the expelling agent and active.

Alternatively, the carbohydrate entrapped volatile component-containing composition is a liquid composition that may be used in combination with a wipe substrate to form a wet wipe, or may be a wetting composition for use in combination with a dispersible wet wipe.

Other modifications and variations to the present invention may be practiced by those of ordinary skill in the art without departing from the spirit and scope of the present invention, which is more particularly set forth in the appended claims. It is understood that aspects of the various embodiments may be interchanged in whole or part. In the event of inconsistencies or contradictions between the incorporated references and this application, the information present in this application shall prevail. The preceding description, given by way of example in order to enable one of ordinary skill in the art to practice the claimed invention, is not to be construed as limiting the scope of the invention, which is defined by the claims and all equivalents thereto.

### EXAMPLES

### Comparative Examples

### Thymol Incorporated Directly onto a Substrate

In this experiment, thymol was mixed with alcohol and placed onto a coform substrate. The resultant wipe was tested for zone of inhibition and found to be unstable for extended periods of time.

Five sections of coform material approximately 17 cm by 17 cm were prepared and weighed to determine the amount of thymol needed for loading of thymol at a weight percent of 0.3%, 0.6% and 1.0%. The determined quantity of thymol to provide thymol as a percentage was placed in an excess (39.0 grams) of ethanol to be dissolved. The coform substrates were placed into a plastic bag and the ethanol/thymol mixture was added. Using a cylindrical glass jar rolled on the outside of the sealed plastic bag, the sheets were rolled to evenly express the liquid throughout the coform squares.

The coform was then removed from the bag and placed in a fume hood to allow the ethanol to evaporate off. The remaining dry sheets containing thymol were removed from the hood after one hour and placed in a dry plastic bag. Samples were aged 7 days and tested for anti-bacterial activity.

To determine the anti-bacterial activity of the samples after 7 days, the shake flask test of ASTM E2149-01 “Standard Test Method for Determining the Antimicrobial Activity of Immobilized Antimicrobial Agents Under Dynamic Contact Conditions” was used. Approximately 1 gram samples of thymol-treated and untreated coform substrates were placed in individual capped flasks containing approximately 10⁵ CFU of a Staphylococcus aureus (ATCC #6538) in phosphate buffered saline. Flasks were shaken at maximum speed on a wrist-action shaker for 1 hour. As can be seen in Table 1 below, the anti-bacterial activity was determined according to ASTM protocol. No meaningful reduction was observed for any thymol-treated or untreated samples. The active agent thymol has volatilized off of the sample within the 7 day time period.

TABLE 1
Log₁₀ Reduction of Staphylococcus aureus (ATCC #6538) after
1 hour exposure to 7 day old sample in ASTM E2149-01

| Thymol % Add-on to Sample | Log₁₀ Reduction of Bacteria |
|---|---|
| 1% Thymol | No Reduction |
| 0.6% Thymol | No Reduction |
| 0.3% Thymol | No Reduction |
| 0.0% Thymol | No Reduction |

To further show that the samples with thymol incorporated directly on the substrate lost all efficacy, zone of inhibition testing was also completed. The samples prepared above were cut into 10 mm-diameter circular sections of thymol-treated and untreated material. These samples were placed on a freshly streaked sample of staphylococcus aureus on trypticase soy agar. After 24 hours incubation at 37° C., plates were measured for zones of inhibitions surrounding the samples. To calculate the zone of inhibition, the test material was brought into contact with a known population of test microorganisms on an agar plate for a specified period of time. At the end of the contact time the area of inhibited colony formation around the test material was measured. The size of this area of no growth was a measure of leaching of the antimicrobial agent from the test material. In this procedure, the test material was cut into small discs and placed on an agar plate evenly spread with a test microorganism. The plates were incubated for 24 hours at ideal growth conditions. Following incubation, the diameter of the circle of no growth around the disc was measured. The zone of inhibition was reported as the difference between the sample disc diameter and the average of the measured no growth zone diameters.

Materials and Reagents
Microorganisms: frozen stock of Staphylococcus aureus (ATCC 6538).
Mueller-Hinton agar (MHA) plates or equivalent plated media. Prepare following manufacturer's directions. Store at 4±2° C. Alternatively pre-made plates can be utilized.
Mueller-Hinton broth (MHB) or equivalent liquid media. Prepare following manufacturers directions. Store at 4±2° C. Alternately pre-made media can be utilized.
Sterile cotton swabs or equivalent.
Sterile forceps.
Positive control disc: Vanocymicin susceptibility discs (6 mm), 30 μg/disc (BD and Company; Sparks, Md.).
Test material, cut into 8 mm discs.
Calipers or other measuring device.
Other ancillary lab supplies.

Preparation

Supply Set-Up

1. Label growth media plates appropriately for testing codes.

2. Sterilize test material discs with UV exposure in Laminar flow hood for 15 minutes (both sides of disc), if required.

Inoculum

1. Take appropriate measures to ensure culture purity.

2. Staphylococcus aureus (ATCC 6538) is inoculated from an overnight plate or MHB into 5 ml of sterile MHB in a 35° C. incubator for 18-24 hrs.

3. The overnight culture is then adjusted using MHB to the 0.5 McFarland barium sulphate standards (1×10⁸ CFU/ml) or approximately 0.15 OD with a 0.2 cm light path at 660 nanometer.

4. Discard the cell suspension if it is not used within 30 to 60 minutes after preparation.

Zone of Inhibition Bioassay Procedure

1. Pre-warm, to room temperature, MHA plates. The number of plates required per strain will depend on the number of test materials to be tested and their anticipated zone inhibition diameters; discs should be placed on plates so that zones of inhibition do not overlap.

2. The surface of the plates should be dry. If not, dry the plates (with lids ajar) in a 35° C. incubator for 20-30 minutes just prior to inoculation. There should be no visible droplets of moisture on the surface of the agar or on the lids of the plates when they are inoculated.

3. Moisten a sterile applicator swab in the standardized cell suspension and express any excess moisture by rotating the swab against the glass above the liquid in the tube. Inoculate the entire surface of each plate, inoculating the surface completely in three different directions to ensure uniform growth.

It is recommended that cotton swabs with wooden handles be used for this procedure. Swabs made of synthetic materials do not soak up sufficient suspension to inoculate the entire surface of the plate. Swabs with plastic handles bend when excess suspension is being expressed and may splatter liquid out of the tube.

4. Repeat step 10.3 to inoculate additional plates as needed.

5. Store the inoculated plates at room temperature for 10-15 minutes to allow the medium to absorb the moisture from the inoculum.

6. Apply discs of test material to the surface of the inoculated medium with a sterile forceps and tap them to ensure that they are in complete contact with the agar surface. A positive control (vancomycin disc) and negative control (uncoated disc) should be used on each plate.

7. Invert the inoculated plates and incubate them at 35° C. for 18-24 hours.

8. Examine the plates from the back, viewed against a black background and illuminated with reflected light. With calipers, measure the diameter of each zone of inhibition to the nearest whole millimeter.

### Calculation

Zone of Inhibition=diameter of no growth area−diameter of disc

As can be seen in Table 1 below, no meaningful reduction was observed for any thymol-treated or untreated samples. The active agent thymol has volatilized off of the samples including thymol within the 7 day time period, losing all efficacy.

TABLE 2
Zone of Inhibition Results of 7 day old sample plated with Staphylococcus
aureus (ATCC #6538)

| | Observed Zone |
|---|---|
| Thymol % Add-on to Sample | of Inhibition (mm) |
| 1% Thymol | 0 mm |
| 0.6% Thymol | 0 mm |
| 0.3% Thymol | 0 mm |
| 0.0% Thymol | 0 mm |

### EXAMPLES

The following non-limiting examples are provided to further illustrate the delivery composition.

First, exemplary Delivery Compositions 1 and 2 were created. A sucrose blend was melted on a stove top to prepare a carbohydrate matrix. An active agent was added to the molten sucrose to provide a 2% loading of thymol. After the mixture was melted, analysis was performed to determine the amount of thymol retained within the sucrose. Delivery Composition 1 contained approximately 0.72 wt. % thymol and Delivery Composition 2 contained approximately 0.74 wt. % thymol. Both Delivery Compositions 1 and 2 were placed in a well in a media with a plate inoculated with Staphylococcus aureus (ATCC #6538) and a zone of inhibition for each sample was measured as described above. Results can be found below in Table 3.

Additionally, exemplary Delivery Compositions 3 and 4 were created. A sucrose blend was melted on a stove top to prepare a carbohydrate matrix. An active agent was added to the molten sucrose to provide a 2% loading of thymol. Then, the mixture was infused with an expelling agent, carbon dioxide. After the mixture was hardened, analysis was performed to determine the amount of thymol retained within the sucrose. Delivery Composition 1 contained approximately 1.9 wt. % thymol and Delivery Composition 2 contained approximately 1.89 wt. % thymol. Both Delivery Compositions 3 and 4 were placed in a well in a media with a plate inoculated with plated with Staphylococcus aureus (ATCC #6538) and a zone of inhibition for each sample was measured as described above. Results can be found below in Table 3.

Finally, a 99% thymol powder was obtained to ascertain its zone of inhibition without a sugar matrix. Various amounts of the thymol powder was placed in a well in a media with a plate inoculated with plated with Staphylococcus aureus (ATCC #6538) and a zone of inhibition for each sample was measured as described above. Results can be found below in Table 3.

TABLE 3
Zone of Inhibition Results Delivery Composition Samples compared
with 99% Pure Thymol

| | Weight | Zone |
|---|---|---|
| Type | (grams) | (mm) |
| Delivery Composition 1 | 0.1498 | 8 |
| Delivery Composition 2 | 0.1591 | 9 |
| Delivery Composition 3 | 0.1574 | 14 |
| Delivery Composition 4 | 0.1506 | 13 |
| 99% Pure Thymol | 0.0045 | 12 |
| 99% Pure Thymol | 0.0018 | 10 |
| 99% Pure Thymol | 0.0789 | 12 |
| 99% Pure Thymol | 0.0353 | 13 |
| 99% Pure Thymol | 0.1609 | 15 |

As can be seen by the results above in Table 3, 99% thymol powder (neat) had a zone of inhibition of 10-15 mm after 1 day. This corresponds with the Delivery Compositions 1-2 and Delivery Compositions 3-4 which had zone of inhibition of 8-9 mm and 13-14 mm respectively. Encapsulating the thymol in the carbohydrate matrix retains the activity of the thymol to disinfect surfaces.

As referenced above, Delivery Compositions 3 and 4 containing a carbohydrate matrix encapsulating both an active agent, thymol, and carbon dioxide, had a 13-14 mm zone of inhibition. This is more similar to the zone of inhibition for pure thymol. Adding an expelling agent to the delivery composition provided a much greater zone of inhibition than the one noticed with only sugar entrapped thymol. This increased the area for zone of inhibition of the delivery composition by approximately 50%. The expelling agent assists with spraying and distributing the disinfectant to a much greater area; and leads to a much greater zone of inhibition for the active.

Having described the disclosure in detail, it will be apparent that modifications and variations are possible without departing from the scope of the disclosure defined in the appended claims.

What is claimed is:

1. A substantially dry substrate for delivering an active agent, the dry wipe comprising:
a substrate; and
a composition comprising:
a carbohydrate matrix;
a volatile active agent entrapped within the carbohydrate matrix; and
an expelling agent entrapped within the carbohydrate matrix.

2. The substantially dry substrate of claim 14 wherein the composition comprises from about 0.01% by weight of the delivery composition to about 10% by weight of the delivery composition of the active agent.

3. The substantially dry substrate of claim 14 wherein the volatile active agent is thymol.

4. The substantially dry substrate of claim 14 wherein the carbohydrate matrix is formed from a simple or complex carbohydrate selected from glucose, fructose, sucrose, lactose, corn syrup, and mixtures thereof.

5. The substantially dry substrate of claim 14 wherein the expelling agent is a gas selected from carbon dioxide, nitric oxide, air, hydrogen sulfide, and nitrogen.

6. The substantially dry substrate of claim 14 wherein the expelling agent is a triggerable expelling agent selected from citric acid and bicarbonate that is triggered by water to produce a gas.

7. The substantially dry substrate of claim 14 wherein the dry substrate is selected from facial tissue, bath tissue, paper towel, or dinner napkin.
